(19) United States
(12) Patent Application Publication
(10) Pub. No.: US 2005/0228477 A1
(43) Pub. Date: Oct. 13, 2005

(54) Topographic coatings and coating methods for medical devices

(75) Inventors: Jeffry J. Grainger, Portola Valley, CA (US); Olexander Hnojewyj, Saratoga, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Xtent, Inc., Menlo Park, CA (US)

(21) Appl. No.: 11/099,418

(22) Filed: Apr. 4, 2005

Related U.S. Application Data
(60) Provisional application No. 60/561,041, filed on Apr. 9, 2004.

(51) Int. Cl. A61F 2/06

(52) U.S. Cl. 623/1.11; 623/1.16; 623/1.42; 427/2.21

(57) ABSTRACT

Medical devices having topographic coatings are provided. The topographic coatings have regions of high and low elevation and may be composed of polymers, metals, ceramics, proteins and other biocompatible materials. Such topographic coatings facilitate the deposition, elution, and protection of therapeutic agents on the medical device, manipulation of the medical device, and other purposes. In particularly preferred embodiments, the medical device comprises a stent for vascular implantation.

### CROSS-REFERENCES TO RELATED APPLICATIONS

The present application is a non-provisional of U.S. Patent Application Ser. No. 60/561,041 (Attorney Docket No. 021629-002600), filed Apr. 9, 2004, the full disclosure of which is incorporated herein by reference.

### BACKGROUND OF THE INVENTION

Coronary stents are tubular scaffolds deployed in stenotic lesions in diseased coronary arteries to maintain the patency of the arterial lumen. Uncoated (or bare metal) coronary stents have suffered from a significant incidence of restenosis, the recurrence of stenotic plaque in the lesion where a stent has been placed. In recent years, coronary stents coated with therapeutic agents such as paclitaxel, rapamycin, or various analogs thereof have shown success in preventing restenosis. In such drug-eluting stents, the therapeutic agent is typically mixed with a durable or bioerodable polymer and applied to the stent by dipping, spraying, or syringe dispensing. However, such techniques suffer from a number of drawbacks. First, these methods are adapted for coating the entire surface or broad regions of the stent, and fail to have the precision to apply a desired pattern at selected locations on the stent surface. Second, such coating methods are not suitable for depositing different carriers or therapeutic agents, different concentrations of therapeutic agents, or coatings of various thicknesses or patterns at different locations on the stent. Further, such coating methods produce a coating that is exposed on the outer surface of the stent and susceptible to damage or removal during assembly, handling, and delivery of the stent to the treatment site. For example, coated stents are typically delivered through a hemostasis valve and a guiding catheter to the desired treatment location. During delivery, the stents may engage the interior of the hemostasis valve and slide against the inner surface of the guiding catheter, damaging or scraping off the stent coating.

To avoid the problems with coating stents, it has been proposed to create pores, channels, or reservoirs in the struts of the stent itself in which therapeutic agents may be deposited. Examples are seen in U.S. patent application Publication Nos. 2003/0068355 and 2004/0039438, and in U.S. Pat. Nos. 6,585,764, 6,527,938, 6,240,616, 6,379,383, 5,972,027, and 6,709,451, which are incorporated herein by reference. Such approaches, however, require drilling, cutting, or etching of the stent struts and/or the use of porous materials to produce the stent, which are complex and costly processes and may adversely affect the strength and performance of the stent.

### BRIEF SUMMARY OF THE INVENTION

The present invention provides implantable medical devices coated with topographic coatings and methods for the manufacture and use thereof. Such topographic coatings are useful for various purposes. First, such coatings may be configured to provide channels, apertures, holes, depressions, reservoirs and other suitable structures to contain therapeutic agents. In addition, such topographic coatings may be configured to protect those regions of the medical device on which therapeutic agents are deposited to prevent damage or removal of therapeutic agents due to contact during assembly, handling, or delivery to a treatment site. Further, such topographic coatings may be used to facilitate manipulation of the medical device by a delivery instrument or catheter.

In a preferred embodiment, the topographic coatings are jet printed onto the surface of the medical device, creating regions of high elevation and low elevation in a predetermined pattern. The topographic coating may be a biocompatible polymer (either durable or bioerodable), metal, ceramic, protein, or other material. The topographic coating may be deposited in various patterns, including in elongate ridges or walls to create linear channels or enclosed reservoirs, in a plurality of discrete bumps or projections, in irregular blobs, in hills and valleys, or in various thicknesses or overlapping layers to create depressions, concavities, or reservoirs. In addition, holes, apertures, depressions, or other reservoirs can be created in the topographic coating after deposition by drilling, heating, etching, or other suitable methods. Regardless of how created, the regions of low elevation may extend only partially through the topographic coating or entirely through it to the surface of the stent or any coating thereon.

One or more therapeutic agents, including anti-restenosis, anti-proliferative, immunosuppressive, antibiotic, thrombolytic, cytotoxic, cystostatic, and other agents, as well as growth factors, DNA, and other substances, may be deposited in the regions of low elevation in the topographic coating. These agents may be deposited with only a solvent which evaporates off, or may be mixed with a durable or bioerodable carrier to provide a delivery matrix for the agent. Different agents and/or different concentrations of the same agent may be deposited in different regions at various locations on the medical device, or within the same region in vertical layers or side-by-side deposits. Further, the topographic coating itself may be mixed, infused or impregnated with a therapeutic agent the same or different than that deposited in the regions of low elevation. Additional layers of polymers, metals, ceramics, proteins, or other materials may be applied to the medical device either over or under the topographic coating and/or therapeutic agent. Such layers may be used to protect the underlying material from damage or removal, to control elution rates of therapeutic agents in the underlying material, to promote adhesion of overlying material to the underlying surface, and other purposes. Such therapeutic agents and other materials may be deposited by spraying, syringe coating, dipping, vacuum deposition, sputtering, and other methods, but preferably such agents and materials are deposited using jet printing, which allows for highly precise deposition in a predetermined pattern coordinated with the pattern of the high and low elevation regions in the topographic coating.

In one embodiment, the medical device is a stent for implantation in a vessel such as a coronary or peripheral artery. The topographic coatings and therapeutic agents of the invention may be applied to any of various known or commercially-available stents, both self-expanding and balloon expandable. In an exemplary embodiment, the topographic coating is disposed on a stent comprising a plurality of separate, unconnected stent segments like those described in copending application Ser. No. 10/738666 (Attorney Docket No. 021629-000510US), filed Dec. 16, 2003, which is incorporated herein by reference. Such segmented stents enable stent length to be customized by the operator in situ using specialized delivery catheters as described in the aforementioned patent application. In some embodiments, these delivery catheters rely upon stent-engaging mechanisms known as “stent valves” mounted near the distal end of the catheter which engage the stent segments to allow the operator to control the position of and spacing between stent segments. Because these stent valves may contact the outer surface of the stent segments, they have the potential to damage or remove any therapeutic agent deposited thereon. The topographic coatings of the invention may be used to minimize such damage by providing a region of higher elevation on the stent surface that may be engaged by the stent valve rather than the stent or coating thereon.

In a first aspect of the invention a stent for deployment in a vessel comprises a cylindrical frame expandable from a contracted shape to an expanded shape and having an outer surface; a topographic layer deposited on at least a portion of the outer surface, the topographic layer forming regions of high elevation and regions of low elevation in a predetermined pattern; and one or more therapeutic agents disposed in the regions of low elevation. At least one of the regions of low elevation may contain a different therapeutic agent than at least one other of the regions of low elevation. The regions of high elevation and low elevation may be dispersed throughout the outer surface, or only on a particular portion thereof. The frame preferably comprises a plurality of struts, at least some of the struts having a region of low elevation thereon.

The regions of low elevation may comprise a plurality of discrete concavities at generally uniform spacing. Alternatively, the regions of low elevation comprise an elongate channel generally aligned longitudinally with each strut. The topographic layer may be formed into two spaced apart ridges to form the channel, or a plurality of independent ridges may be formed, each ridge enclosing a region of low elevation. Preferably, the height of the topographic layer adjacent to the regions of low elevation is higher than a top surface of the therapeutic agent in the regions of low elevation. The topographic layer is a biocompatible material selected from polymers, metals, ceramics, proteins, hydrogels, and crystalline materials.

The topographic layer may contain no therapeutic agent, or it may be mixed or impregnated with a therapeutic agent that elutes from the topographic layer produce a desired therapeutic effect. The topographic layer may be bioerodable, bioabsorable or durable, and may have a coating of a polymer or other suitable material over it to control elution rate of any agent therein. Preferably, in coronary applications, at least about 70%, preferably at least 80%, and more preferably 90% of the therapeutic agent elutes from the regions of low elevation and/or topographic layer within about 30 days. A base layer may optionally be deposited on the outer surface of the frame under the topographic layer to enhance adhesion, to provide biocompatibility, or for other purposes.

In the regions of low elevation, the therapeutic agent may be deposited alone or mixed with a carrier. The carrier may be the same or different material as that used for the topographic layer. Usually, the fame is a metal and the topographic layer is a polymer, although stents made of polymers and other materials, both durable and bioerodable, are possible. The topographic layer may also be a metal or oxide that is sputtered, sintered, or otherwise deposited on the stent surface. The metal may be same or different as that used for the stent. Other materials suitable for the topographic layer include ceramics and proteins, although various other biocompatible materials having appropriate properties for adhesion to the stent may also be used.

The topographic layer may be deposited in a variety of patterns on the stent. In some embodiments, portions of the stent frame remain uncovered by the topographic layer. Further, the regions of low elevation extend only partially through the topographic layer, or entirely through its thickness to the surface of the frame or any coating thereon. The regions of high elevation may comprise dots or bumps in various shapes including cylindrical, dome-shaped, conical, or irregular shapes. Alternatively, the regions of high elevation may comprise elongate ridges or walls. The regions of high elevation are preferably configured to protect the therapeutic agent in adjacent regions of low elevation from contact prior to deployment of the stent. In some embodiments, at least one of the regions of high elevation and low elevation is adapted for engagement by a delivery catheter for manipulation of the stent.

In a further aspect of the invention, a stent delivery system for delivery of stents to a vessel comprises an elongated flexible catheter shaft having a proximal end and a distal end; a plurality of expandable stents positionable near the distal end, the stents comprising an outer surface and a topographic layer deposited on the outer surface forming a plurality of regions of high elevation and regions of low elevation; a deployment mechanism for releasing the stents from the catheter; and a stent-engaging structure near the distal end configured to engage the stents to control the position thereof on the catheter shaft.

In a preferred embodiment, a therapeutic agent is deposited in the regions of low elevation and wherein the regions of high elevation of the topographic layer protect the therapeutic agent prior to deployment of the stent. In a further aspect, at least one of the regions of high elevation and low elevation is configured to be engaged by the stent-engaging structure for controlling the position of the stent. In these embodiments, the regions of high elevation are configured to reduce contact between the stent-engaging structure and the therapeutic agent. In some embodiments, the regions of high elevation are configured to be deformed, cut, or flattened when engaged by the stent-engaging structure.

In another aspect of the invention, a method of processing a stent comprises jet printing a topographic layer on an outer surface of the stent in a predetermined pattern, the topographic layer having regions of high elevation and regions of low elevation; and depositing a first therapeutic agent in the regions of low elevation. The step of depositing preferably comprises jet printing the first therapeutic agent in the regions of low elevation. Further, a second therapeutic agent may be deposited in selected regions of low elevation. In some cases, the second therapeutic agent is jet printed in the selected regions of low elevation.

The topographic layer may be jet printed in various patterns on the stent. In one embodiment, the predetermined pattern comprises at least one elongated ridge. The pattern may further comprise spaced-apart elongated ridges forming at least one channel therebetween, the first therapeutic agent being deposited in the channel. Alternatively, the predetermined pattern may comprise a plurality of bumps or dots at predetermined spacing.

In addition to stents, the principles of the invention may be applied to a wide variety of medical devices on which a therapeutic agent may be coated or which might benefit from a topographic coating for manipulation, surface protection or other purposes. Such devices include heart valve prostheses, annuloplasty rings, orthopedic implants, vascular grafts, embolic coils, anastomosis devices, and others.

Further aspects of the nature and advantages of the invention are set forth in the following detailed description to be taken in conjunction with the drawings.

### BRIEF DESCRIPTION OF THE DRAWINGS

FIG. 1 is a side elevational view of a stent having a topographic coating according to the invention.

FIG. 2 is a close-up view of a portion of the stent of FIG. 1.

FIGS. 3A-3B are a top view and transverse cross-section, respectively of a strut in the stent of FIG. 1 in a first embodiment thereof.

FIGS. 4A-4B are a top view and transverse cross-section, respectively of a strut in the stent of FIG. 1 in a second embodiment thereof.

FIGS. 5A-5B are a top view and transverse cross-section, respectively of a strut in the stent of FIG. 1 in a third embodiment thereof.

FIG. 6 is a close-up view of a portion of a stent having a topographic coating according to the invention in a further embodiment thereof.

FIGS. 7A-7B are a top view and transverse cross-section, respectively of a strut in the stent of FIG. 6 in a first embodiment thereof.

FIGS. 8A-8B are a top view and transverse cross-section, respectively of a strut in the stent of FIG. 6 in a second embodiment thereof.

FIGS. 9A-9B are a top view and transverse cross-section, respectively of a strut in the stent of FIG. 6 in a third embodiment thereof.

FIG. 10 is a close-up view of a portion of a stent having a topographic coating according to the invention in another embodiment thereof.

FIGS. 11-14 are side cross-sectional views of a strut in the stent of FIG. 10, showing various embodiments of the topographic coating thereon.

FIG. 15 is an oblique view of a strut in a stent according to the invention showing a further embodiment of a topographic coating and inner surface coating thereon.

FIGS. 16-19 are side cross-sectional views of the strut of FIG. 15 showing various embodiments of the topographic coating and an inner surface coating.

FIG. 20 is a close-up view of a portion of a stent according to the invention schematically illustrating a coating comprising a plurality of therapeutic agents in different regions of the stent.

FIGS. 21-22 are side elevational views of a stent showing two additional embodiments of a topographic coating according to the invention.

FIG. 23 is a schematic of a jet printing apparatus for coating a stent with a topographic coating according to the invention.

FIG. 24 is a print head and stent holding apparatus for coating the inner surface of a stent using the apparatus of FIG. 23.

### DETAILED DESCRIPTION OF THE INVENTION

Stents to which the principles of the invention may be applied include any of the various known or commercially available coronary or peripheral stents. Suitable stents and delivery devices are further described in copending applications Ser. No. 10/306813 (Attorney Docket No. 021629-000320US), filed Nov. 27, 2002; Ser. No. 10/412714 (Attorney Docket No. 021629-000330US), filed Apr. 10, 2003; Ser. No. 10/637713 (Attorney Docket No. 021629-000340US), filed Aug, 8, 2003; Ser. No. 10/624451 (Attorney Docket No. 021629-000400US), filed Jul. 21, 2003; Ser. No. 10/738666 (Attorney Docket No. 021629-000510US), filed Dec. 16, 2003; Ser. No. 10/458062 (Attorney Docket No. 021629-001800US), filed Jun. 9, 2003; Ser. No. 10/686507 (Attorney Docket No. 021629-001900US), filed Oct. 14, 2003; Ser. No. 10/686025 (Attorney Docket No. 021629-002000US), filed Oct. 14, 2003; Ser. No. 10/687532 (Attorney Docket No. 021629-002100US), filed Oct. 15, 2003; Ser. No. 10/46466 (Attorney Docket No. 021629-002200US), filed Dec. 23, 2003; and Ser. No. 10/794,405 (Attorney Docket No. 021629-002400US), filed Mar. 3, 2004, all of which are hereby incorporated fully by reference.

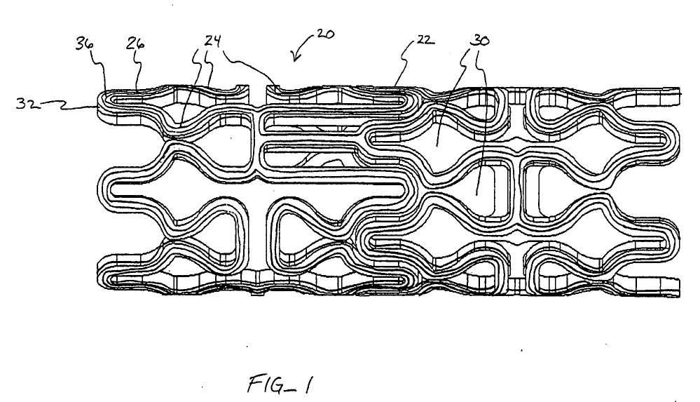

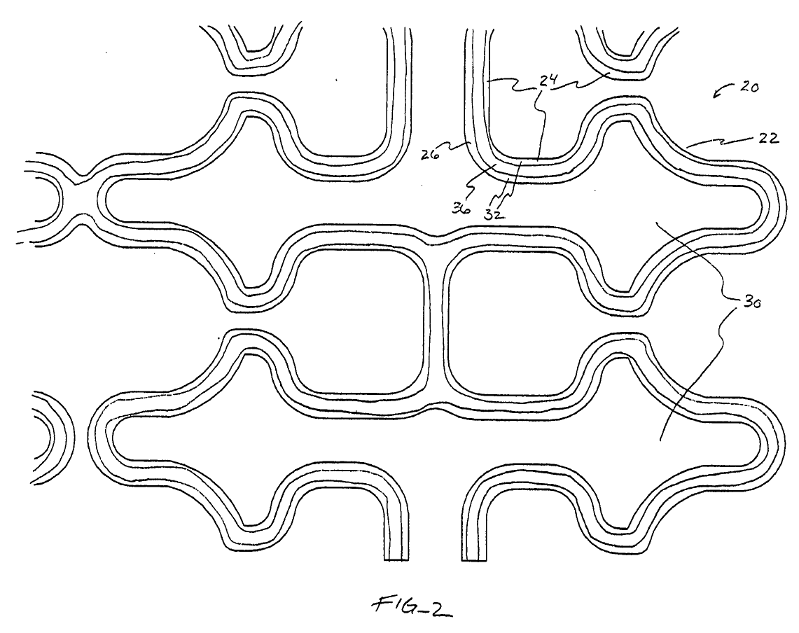

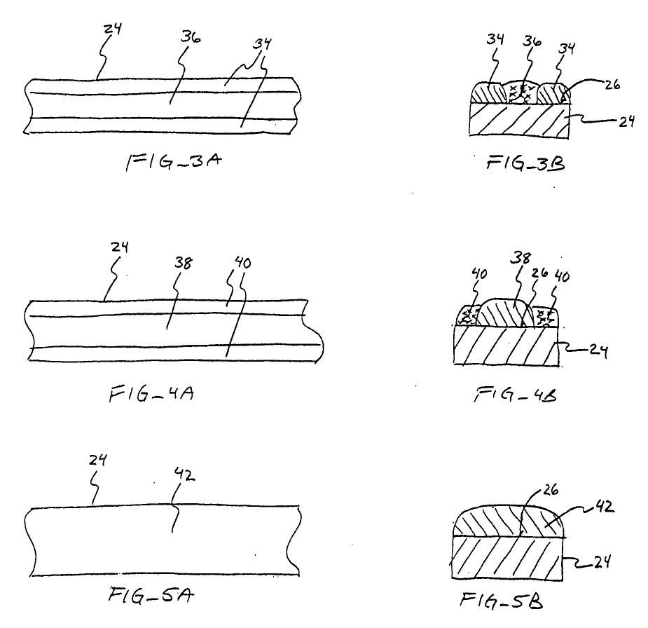

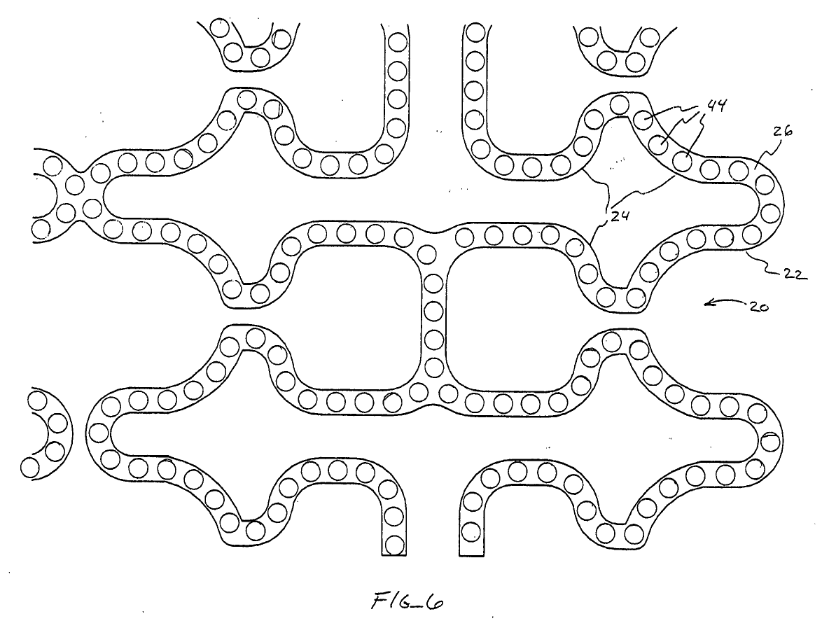

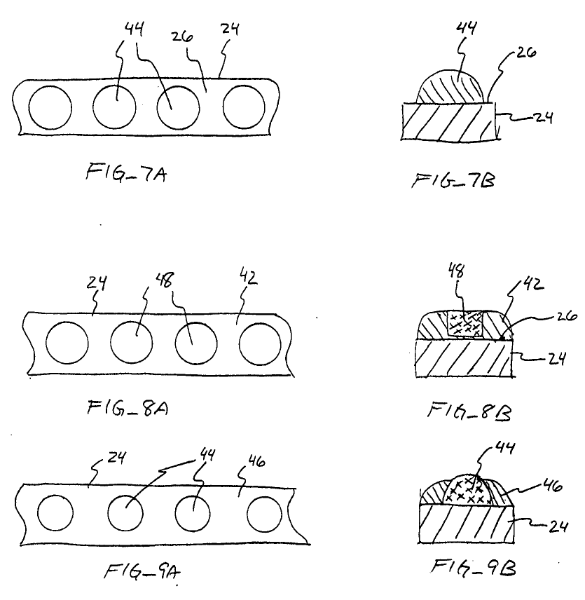

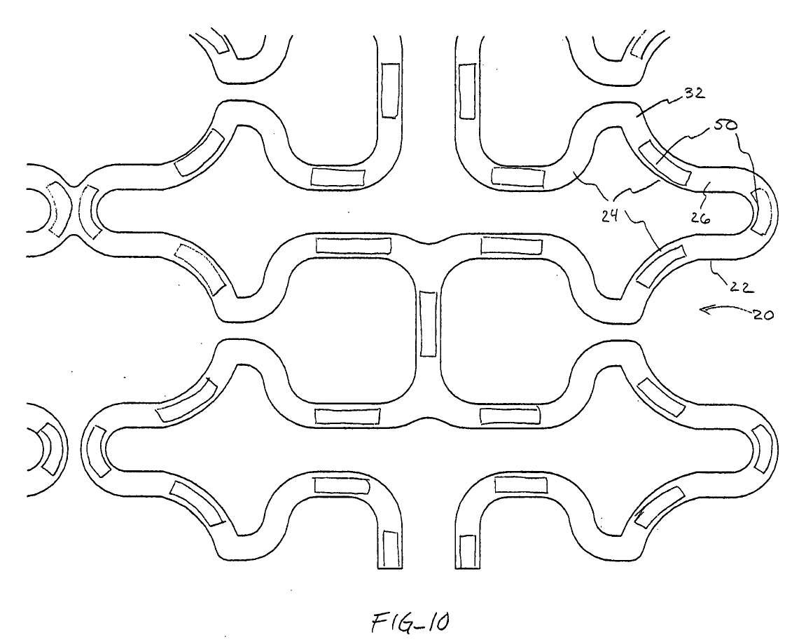

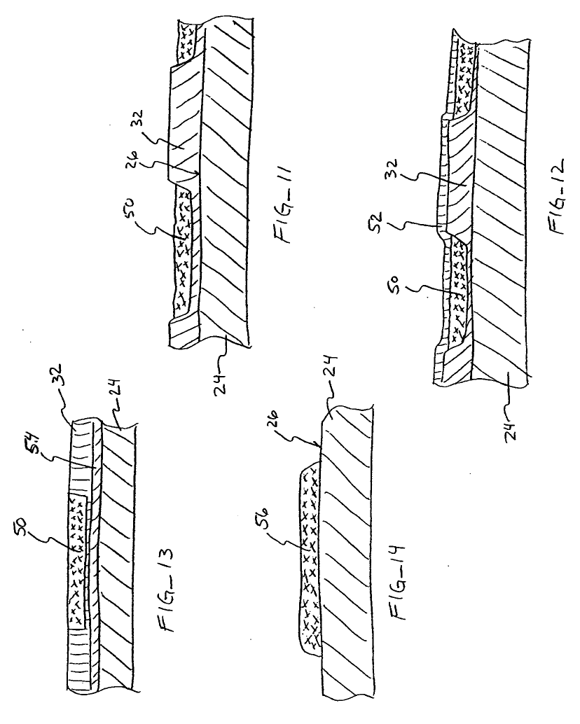

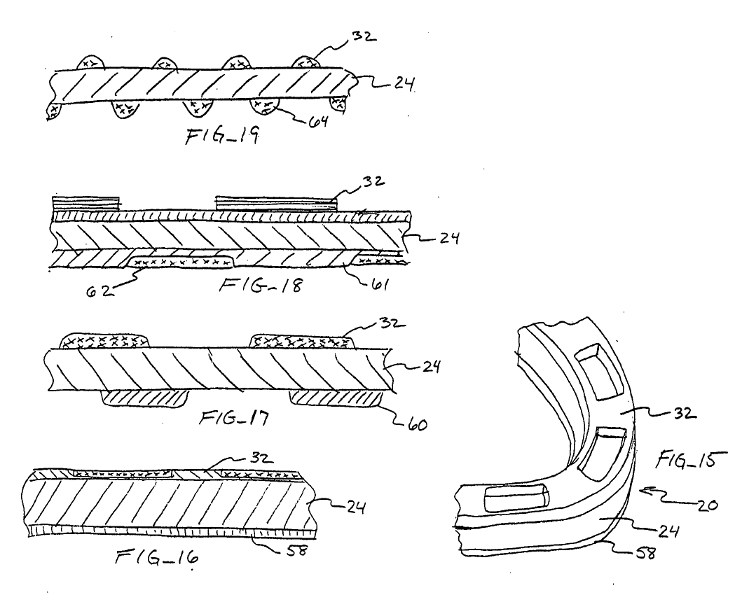

Referring to FIGS. 1-2, in an exemplary embodiment, a stent 20 comprises a cylindrical frame 22 having a plurality of struts 24. Struts 24 have an outer surface 26 and an inner surface (not visible in FIG. 1) on the opposite side thereof facing the interior of frame 22. Struts 24 are disposed in a pattern of axial, circumferential, curved and oblique segments which define openings 30 communicating with the interior of frame 22. Frame 20 may be constructed of various biocompatible metals or polymers, may have either an open cell or closed cell design, and may be either balloon expandable or self-expanding. Stent 20 will have dimensions suitable for the anatomical region in which the stent is used; in one embodiment suitable for coronary use, frame 22 has a length of about 2-60 mm, and diameter of about 2-6 mm, while struts 24 have a radial thickness of about 0.001-0.006″, more preferably 0.002-0.004″, and a circumferential width of about 0.002″-0.006″. If a segmented stent design is employed, stent 20 will comprise a plurality of unconnected stent segments having the construction of frame 22, each segment being about 2-10 mm in length. In this embodiment, multiple stent segments may be deployed together to stent a particular lesion up to 60 mm or more in length. It will be appreciated that the principles of the invention are equally applicable to single-piece stents, interconnected stent segments, and other designs.

On outer surface 26 a topographic layer 32 is deposited in a predetermined pattern to form regions of high elevation and regions of low elevation relative to outer surface 26. In one embodiment, shown in FIGS. 3A-3B, topographic layer 32 is deposited to form two parallel ridges or walls 34 with a channel 36 therebetween. Alternatively, 3, 4 or more generally parallel ridges may be deposited on outer surface 26 to form 2, 3 or more channels therebetween in which therapeutic agents may be deposited. The topographic layer may be a polymer, metal, ceramic, protein, or other suitable material that adheres to struts 24, is expandable with frame 22 without excessive cracking or loss of adherence, and provides suitable structural characteristics to contain and protect the therapeutic agent. Topographic layer 32 may be permanent, semi-permanent, or bioerodable, and may be impregnated or mixed with a therapeutic agent that can diffuse into the vessel wall and/or blood stream at a desired rate. Ridges 34 each have a width of less than half the width of struts 24, preferably each being about 10-40% and more preferably 20-30% of the width of struts 24, with channel 36 being about 10-80%, preferably 20-60%, of the width of struts 24. The thickness of topographic layer 32 will usually be less than 50% of the radial thickness of struts 24, preferably less than 25% of the radial thickness of struts 24, and more preferably less than 10% of the thickness of struts 24. In an exemplary embodiment, topographic layer 32 has a thickness of about 0.0001-0.0010″, preferably 0.0002-0.0006″ at its thickest point.

An underlayer or primer of a polymer such as Teflon, parylene or other suitable material may also be deposited on outer surface 26 under topographic layer 32 to improve adherence or for other purposes. In one embodiment, stent 20 has a layer of parylene less than 0.0005″, preferably about 0.0001-0.0003″, in thickness on outer surface 26.

A therapeutic agent may be deposited in channel 36. Preferably, the therapeutic agent is deposited to an elevation no higher than and preferably less than that of walls 34 so that it is protected from damage during handling and delivery to the treatment site via catheter. The therapeutic agent may be mixed or impregnated in a durable or bioerodable polymer matrix, or may be deposited without a carrier. The therapeutic agent may further be coated with polymers or other materials to control its elution rate, protect it from damage during delivery, or other purposes. In a preferred embodiment, the therapeutic agent comprises Rapamycin or an analog thereof such as Biolimus A9, Everolimus, or ABT 578, mixed in a polymeric carrier, either bioerodable (such as polylactic acid) or durable. Preferably the therapeutic agent is applied so that stent 20 has about 10-20 micrograms, preferably about 14-16 micrograms, more preferably 15.6 micrograms, of therapeutic agent per millimeter of stent 20. In an exemplary embodiment, a solution comprising 50 mg drug and 50 mg polymer in 2 ml of acetone with a concentration of 3% solids is used.

In a second embodiment, shown in FIGS. 4A-B, topographic layer 32 is formed in a single ridge 38 on outer surface 26 generally parallel to the struts 24, forming two low elevation regions 40 on either side of ridge 38. A therapeutic agent may then be deposited in either or both low elevation regions 40.

In a third embodiment, shown in FIGS. 5A-5B, topographic layer 32 is formed in a single ridge 42 covering substantially all of outer surface 26. Topographic layer 32 may be impregnated or mixed with a therapeutic agent that elutes from it at a desired rate. Alternatively, reservoirs, depressions, concavities, holes or other regions of low elevation may be formed in ridge 42 by masking during deposition or after deposition by drilling, heating, cutting, etching, or other methods, as illustrated in FIGS. 8A-B below. A therapeutic agent may then be deposited in the low elevation regions. As a further alternative, topographic layer 32 may be used to facilitate manipulation of the stent in a stent delivery catheter, as described more fully below.

Referring now to FIGS. 6-7, in a further embodiment, topographic layer 32 is formed in a plurality of bumps 44 in a predetermined pattern on outer surface 26. In this embodiment, topographic layer 32 may be comprised of a therapeutic agent alone or contained in a carrier or matrix. Bumps 44 may be cylindrical, mound-shaped, disk-shaped, cone-shaped, oblong, square, rectangular or irregularly shaped, and are spaced apart in a predetermined pattern on outer surface 26. Bumps 44 may have a diameter (or transverse dimension) as large as the width of struts 24, or may be smaller, e.g. about 10-90%, more preferably about 25-75%, of the width of struts 24. Bumps 44 may be of various thickness, preferably being about 0.0002-0.0006″ thick. Through the use of jet printing technology, bumps 44 may be as small as one micron in diameter. The density, pattern, shape, or size of bumps 44, or the concentration or type of agent in bumps 44, may be different at different locations on outer surface 26 to create different elution profiles and different therapeutic effects at different points along stent 20. Further, bumps 44 may be disposed in a pattern and with size and thickness to facilitate manipulation by a stent delivery catheter as described below.

In a further embodiment, a second material 46 may be deposited around bumps 44 as illustrated in FIGS. 9A-B. Material 46 may be a polymer, ceramic, metal, protein, drug, or other durable or bioerodable material, and may serve to stabilize and protect bumps 44 from damage or removal, to elute therapeutic agents, to facilitate manipulation of stent 20, or for other purposes.

In a further embodiment, illustrated in FIGS. 8A-8B, topographic layer 32 may comprise a single covering 42 over substantially all of outer surface 26. A plurality of concavities 48 are formed in covering 42 in a desired pattern by masking outer surface 26 prior to deposition of covering 42, or by drilling, melting, cutting, etching or otherwise forming concavities 48 after deposition of covering 42. Concavities 48 may then be filled entirely or partially with a therapeutic agent by means of microjet printing, dipping, spraying, syringe dispensing, vacuum deposition or other suitable technique.

FIGS. 10-13 illustrate further exemplary embodiments of topographic layer 32 on a stent 20. In the embodiment of FIG. 11, topographic layer 32 covers substantially all of top surface 26 on struts 24, and has a plurality of elongated concavities 50 formed therein. Concavities 50 may have various shapes, including rectangular, oval, square, round, or irregular shape. In a preferred embodiment, concavities 50 extend only partially through the thickness of topographic layer 32. A therapeutic agent may be deposited in concavities 50, with or without a carrier or matrix. A shown in FIG. 12, a top layer 52 may optionally be deposited on top of topographic layer 32 and/or the therapeutic agent in concavities 50 which may be a durable or bioerodable material to control the elution rate of the therapeutic agent or protect it from damage during delivery. In a further alternative, shown in FIG. 13, an underlayer 54 may be deposited on outer surface 26 before deposition of topographic layer 32 to enhance adhesion.

In yet another alternative, shown in FIG. 14, topographic layer 32 comprises a plurality of discreet patches 56 deposited on outer surface 26. Patches 56 may be of various shapes including rectangular, square, round, oval, or irregular. Patches 56 may be placed at predetermined patterns and spacings which may vary at different point along struts 24. Further, patches 56 may have various sizes and may be composed of different therapeutic agents or different concentrations of agent at different places along struts 24. Again, an underlayer of polymer or other suitable material may be deposited on outer surface 26 prior to deposition of patches 56 to enhance adhesion or for other purposes.

Referring now to FIGS. 15-19, in a further embodiment, struts 24 of stent 20 may further include an inner layer 58 in a selected pattern on inner surface 60, alone or in addition to topographic layer 32 on outer surface 26. Inner layer 58 may comprise a single layer of uniform thickness (FIG. 16), a plurality of discreet patches 60 (FIG. 17), a layer 61 with a plurality of concavities, channels, or holes 62 in it (FIG. 18), a plurality of bumps 64 in a desired pattern (FIG. 19), or any of the various other configurations described above with respect to topographic layer 32. Inner layer 58 may contain a therapeutic agent alone or with a carrier, which may be the same or different as those used in topographic layer 32. For example, inner layer 58 may include a thrombolytic agent, while topographic layer 32 includes an anti-proliferative agent.

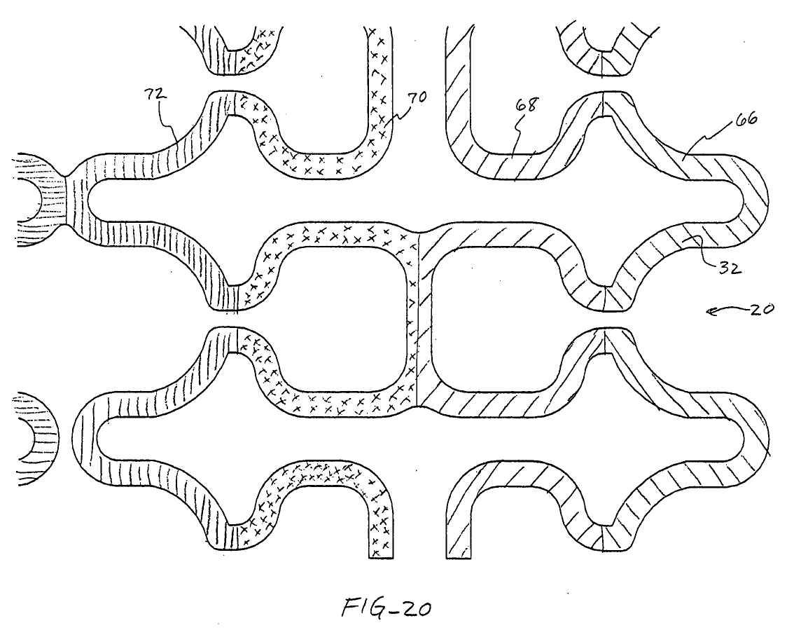

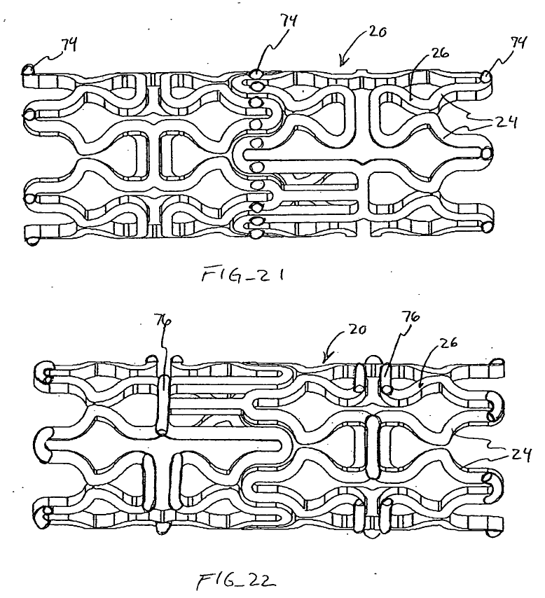

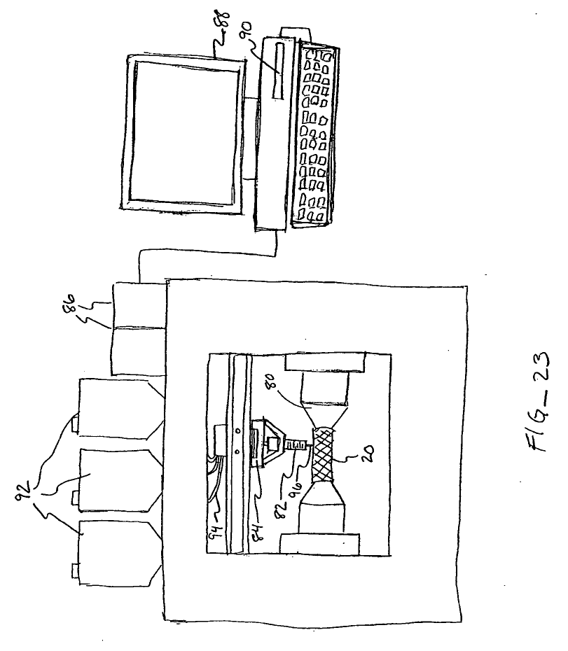

FIG. 20 illustrates a further embodiment in which topographic layer 32 comprises therapeutic agents of various types or concentrations at various regions along stent 20. For example, region 66 contains a different agent than region 68, which contains a different agent than region 70, which contains a different agent than region 72. Some or all of the therapeutic agents in each region may be mixed or impregnated in a durable or bioerodable carrier and may be covered by an additional layer for controlling elution rates. Further, topographic layer 32 in each region may have any of the configurations and patterns of high and low elevation described above in connection with other embodiments, and such patterns and configurations may be different in each region. Such regions may be applied in various patterns on stent 20, including circumferential bands, axial stripes, diagonal stripes, or discrete dots or bumps. Stent 20 may have different therapeutic agents at different regions around its circumference. Each strut or portion of a strut may even be coated with a different therapeutic agent.

FIGS. 21-22 illustrate additional embodiments of a stent 20 having a topographic layer deposited so as to create circumferential regions of higher elevation to protect any coating on the stent and to facilitate manipulation of stent 20 during deployment by a delivery catheter. In FIG. 21, a series of bumps 74 are deposited in circumferential rows at various points along stent 20. In FIG. 22, elongated ridges 76 conforming to the shape of struts 24 are deposited around the circumference of stent 20 at a series of spaced-apart axial locations. In either case, the topographic layer may be deposited on top of a coating of therapeutic agent on the stent, or it may be deposited directly on the stent (or primer coat thereon) before the therapeutic agent is applied to the stent. Bumps 74 or ridges 76 may serve as bumpers to maintain spacing between outer surface 26 and the inner wall of the delivery catheter, sheath, or guiding catheter through which the stent is delivered, thereby protecting the coating of therapeutic agent on the stent, the surface of which lies a at a lower elevation than such bumps or ridges. In addition, bumps 74 or ridges 76 may be used for engagement by a stent valve or other stent-engaging mechanism in the delivery catheter for manipulating and positioning the stent therein, as described in copending application Ser. No. 10/637,713, filed Aug. 8, 2003, which is incorporated herein by reference for all purposes. To enable such engagement, bumps 74 or ridges 76 will have a thickness of about 0.0005-0.002″, more preferably 0.001-0.0015″. Such bumps or ridges may be configured to be deformed or partially removed by engagement with such a stent-engaging mechanism or otherwise during delivery.

In a preferred embodiment, the topographic coatings and therapeutic agents of the invention are deposited on stent 20 using microjet dispensing (or jet printing) technology. Such technology is used in a variety of high precision printing and dispensing applications, most commonly in ink jet printers. Microjet dispensing has also been used for dispensing of liquid metals such as solder, chemicals, adhesives, electronic materials, drugs, proteins, DNA, polymers, cells, growth factors and other materials. See, e.g., Cooler et al., Applications of Ink-Jet Printing Technology to BioMEMS and Microfluidic Systems, Proceedings, SPIE Conference on Microfluidics and BioMEMS, October 2001). Exemplary patents describing the construction and use of microjet dispensing systems include U.S. Pat. Nos. 5,772,106, 4,812,856, 5,053,100, 3,683,212, 5,658,802, 6,367,925, 6,188,416, 6,645,547, 6,378,988, 5,444,467, which are incorporated herein by reference.

FIG. 23 schematically illustrates an apparatus for depositing a topographic layer and/or therapeutic agents on a stent in any of the patterns and configurations described above. A stent 20 is held in a stent holder 80 which is capable of rotating stent 20 about its longitudinal axis. A print head assembly 82 is mounted to a positioner 84 capable of moving print head assembly 82 along the X, Y, and Z axes. Print head assembly 82 has a print head 96 which may be any suitable microjet print head as described in U.S. Pat. Nos. 5,772,106, 4,812,856, 5,053,100, 3,683,212, 5,658,802, 6,367,925, 6,188,416, 6,645,547, 6,378,988, 5,444,467, which are incorporated herein by reference. Controllers 86 are electronically coupled to stent holder 80 and positioner 84 to control the movement and speed thereof. Materials for the topographic layer and any therapeutic agents to be deposited are contained in supply containers 92, which are coupled via tubes 94 to print head assembly 82. Supply containers 92 may contain a plurality of different therapeutic agents, polymers, metals, ceramics, proteins or other materials to be deposited at various locations on stent 20. A computer 88 is coupled to controllers 86 for providing program instructions thereto. Data files regarding the design of stent 20, desired pattern for the topographic layer, the type and location of therapeutic agents and other materials to be deposited thereon, and other required information may be input through data storage medium drive 90, which may be a CD, DVD, hard disk or other suitable drive. In this way, a topographic layer, along with therapeutic agents, overlayers, and underlayers may be deposited in a variety of patterns on stent 20 with precision, speed, and little wastage of material.

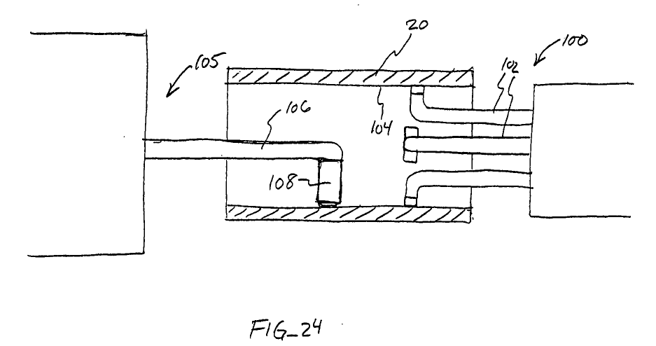

In order to apply a coating or pattern of topographic features to the inner wall of a stent, a stent holding apparatus and print head assembly as shown in FIG. 24 may be used in the microjet printing assembly of FIG. 23. Stent holding apparatus 100 is configured to hold stent 20 from one end thereof, and includes a plurality of jaws 102 that extend into the interior of stent 20 and press outwardly to engage inner wall 104. Printhead assembly 105 is arranged horizontally and has a long neck portion 106 configured to extend into the interior of stent 20, and a laterally facing head 108 for depositing materials on inner wall 104. In this embodiment, stent holding apparatus 100 is capable of rotating stent 20 about its longitudinal axis while print head assembly 105 is movable axially and in the radial direction relative to stent 20.

The topographic coatings of the invention may be deposited using various jet printing techniques, including dot-to-dot (DTD), wherein one or more discrete dots are deposited at a preselected spacing, and printing on the fly (POF), wherein the printhead and/or stent are moved relative to one another as dots of coating material are dispensed at a constant rate, thereby forming a continuous elongated or linear shape, either straight or curved. Such POF techniques may be used to create topographic layer 32 in ridges, walls, channels, patches, and other elongated shapes as described above. Further, topographic layers of greater thickness may be created by dispensing multiple layers on top of one another.

In a further aspect of the invention, metals, polymers or other suitable materials may be deposited over a removable, meltable, or dissolvable substrate to create a stent or other bioprosthesis itself For example, a removable mandrel or tubular substrate of a dissolvable or meltable polymer may be placed in the jet printing apparatus of FIG. 23 and one or more layers of metal or polymer may be deposited on the substrate in a desired pattern to form a stent having a desired strut shape and cell design. Multiple layers can be deposited to build up the desired strut thickness, which may vary at various positions along the stent if desired. Different materials may be used at different locations along the stent as well. The stent may be constructed partially or entirely of a porous material (polymer or metal) in which therapeutic agents are mixed or embedded. Alternatively, a layer of therapeutic agents, polymers, or other materials may be sandwiched between layers of metal or polymer or applied to the outer surface once the stent is formed. The stent may then be removed from the jet printing apparatus and the tubular substrate removed by heating, dissolving by immersion in a liquid bath, mechanical drilling or cutting, or other suitable method.

While jet printing is the preferred technique for depositing the topographic layers and therapeutic agent coatings of the invention, it will be understood that various other techniques also may be used, alone or in conjunction with jet printing. Such techniques include, dipping, spraying, syringe dispensing, masking and etching, ion deposition, vapor deposition, vacuum deposition, photolithography, sterolithography, sputtering, sintering, and other techniques.

While the above is a complete description of the preferred embodiments of the invention, various alternatives, modifications, substitutions, and equivalents are possible without departing from the scope thereof, which is defined by the claims.

1. A stent for deployment in a vessel comprising:
a cylindrical frame expandable from a contracted shape to an expanded shape and having an outer surface;
a topographic layer deposited on at least a portion of the outer surface, the topographic layer forming regions of high elevation and regions of low elevation in a predetermined pattern; and
one or more therapeutic agents disposed in the regions of low elevation.

2. The stent of claim 1 wherein the regions of low elevation comprise a plurality of discrete concavities at generally uniform spacing.

3. The stent of claim 1 wherein at least one of the regions of low elevation contains a different therapeutic agent than at least one other of the regions of low elevation.

4. The stent of claim 1 wherein the regions of high elevation and low elevation are dispersed throughout the outer surface.

5. The stent of claim 1 wherein the frame comprises a plurality of struts, at least some of the struts having a regions of low elevation thereon.

6. The stent of claim 5 wherein the regions of low elevation comprise an elongate channel generally aligned longitudinally with each strut.

7. The stent of claim 6 wherein the topographic layer is formed into two spaced apart ridges to form the channel.

8. The stent of claim 1 wherein the regions of high elevation comprise a plurality of independent ridges, each ridge enclosing a region of low elevation.

9. The stent of claim 1 wherein the height of the topographic layer adjacent to the regions of low elevation is higher than a top surface of the therapeutic agent in the regions of low elevation.

10. The stent of claim 1 wherein the topographic layer is a material selected from polymers, metals, ceramics, proteins, hydrogels, and crystalline materials.

11. The stent of claim 1 wherein the topographic layer contains no therapeutic agent.

12. The stent of claim 1 wherein the topographic layer contains a therapeutic agent.

13. The stent of claim 1 wherein the topographic layer is bioabsorbable.

14. The stent of claim 1 wherein no less than about 90% of the therapeutic agent elutes from the regions of low elevation within about 30 days.

15. The stent of claim 1 further comprising an elution control layer deposited on the therapeutic agent.

16. The stent of claim 1 further comprising a base layer deposited on the outer surface of the frame under the topographic layer.

17. The stent of claim 1 wherein the therapeutic agent is mixed with a carrier in the regions of low elevation.

18. The stent of claim 17 wherein the carrier is a different material than the topographic layer.

19. The stent of claim 1 wherein the frame is a metal and the topographic layer is a polymer.

20. The stent of claim 1 wherein the frame is a first metal and the topographic layer is a second metal.

21. The stent of claim 1 wherein the topographic layer is deposited at a plurality of predetermined locations on the outer surface of the frame, the frame being uncovered by the topographic layer except at the predetermined locations.

22. The stent of claim 1 wherein the regions of low elevation extend only partially through the topographic layer.

23. The stent of claim 1 wherein the regions of low elevation extend entirely through the topographic layer to the outer surface of the frame.

24. The stent of claim 1 wherein the regions of high elevation comprise generally cylindrical, dome-shaped, conical, or irregularly shaped bumps.

25. The stent of claim 1 wherein the regions of high elevation comprise elongate ridges or walls.

26. The stent of claim 1 wherein the regions of high elevation are configured to protect the therapeutic agent in adjacent regions of low elevation from contact prior to deployment of the stent.

27. The stent of claim 1 wherein at least one of the regions of high elevation and low elevation is adapted for engagement by a delivery catheter for manipulation of the stent.

28. A stent delivery system for delivery of stents to a vessel comprising:
an elongated flexible catheter shaft having a proximal end and a distal end;
a plurality of expandable stents positionable near the distal end, the stents comprising an outer surface and a topographic layer deposited on the outer surface forming a plurality of regions of high elevation and regions of low elevation;
a deployment mechanism for releasing the stents from the catheter; and
a stent-engaging structure near the distal end configured to engage the stents to control the position thereof on the catheter shaft.

29. The stent delivery system of claim 28 wherein a therapeutic agent is deposited in the regions of low elevation and wherein the regions of high elevation of the topographic layer protect the therapeutic agent prior to deployment of the stent.

30. The stent delivery system of claim 28 wherein at least one of the regions of high elevation and low elevation is configured to be engaged by the stent-engaging structure for controlling the position of the stent.

31. The stent delivery system of claim 29 wherein the regions of high elevation are configured to reduce contact between the stent-engaging structure and the therapeutic agent.

32. The stent delivery system of claim 28 wherein the regions of high elevation are configured to be deformed, cut, or flattened when engaged by the stent-engaging structure.

33. A method of processing a stent comprising:
jet printing a topographic layer on an outer surface of the stent in a predetermined pattern, the topographic layer having regions of high elevation and regions of low elevation; and
depositing a first therapeutic agent in the regions of low elevation.

34. The method of claim 33 wherein depositing comprises jet printing the first therapeutic agent in the regions of low elevation.

35. The method of claim 33 further comprising depositing a second therapeutic agent in selected regions of low elevation.

36. The method of claim 35 wherein depositing the second therapeutic agent comprises jet printing the therapeutic agent in the selected regions of low elevation.

37. The method of claim 33 wherein the predetermined pattern comprises at least one elongated ridge.

38. The method of claim 33 wherein the predetermined pattern comprises spaced apart elongated ridges forming at least one channel therebetween, the first therapeutic agent being deposited in the channel.

39. The method of claim 33 wherein the predetermined pattern comprises a plurality of bumps at predetermined spacing.
